(19) United States
(12) Patent Application Publication
(10) Pub. No.: US 2020/0055044 A1
(43) Pub. Date: Feb. 20, 2020

(54) METHODS AND SYSTEMS FOR MICRO PLATELET FUNCTION TESTING USING AN INTEGRATED MINIATURIZED PLATELET FUNCTION ANALYZER

(71) Applicant: Atantares Corp., Arlington, MA (US)

(72) Inventors: Qi QIN, Arlington, MA (US); Aniruddh SARKAR, Cambridge, MA (US); Xin ZHAO, Chestnut Hill, MA (US); Kumaran KOLANDAIVELU, Jamaica Plain, MA (US)

(21) Appl. No.: 16/346,746

(22) PCT Filed: Nov. 2, 2017
(86) PCT No.: PCT/US2017/059658

§ 371 Date: May 1, 2019

Related U.S. Application Data
(60) Provisional application No. 62/416,247, filed on Nov. 2, 2016.

(51) Int. Cl. B01L 3/00; G01N 33/49; G01N 27/07; G01N 27/08

(52) U.S. Cl. CPC B01L 3/502715; B01L 3/50273; G01N 33/49; G01N 27/07; G01N 27/08; B01L 2400/0475; B01L 2300/16; B01L 2300/047; B01L 2200/16; B01L 2200/0647; B01L 2300/0645

(57) ABSTRACT

Disclosed are methods and devices for platelet function analysis. An impedance-based micro platelet function test device comprises a pair of micro-fabricated 3D electrodes for measuring an electrical impedance between the pair electrodes during a platelet function test, and a microfluidic chamber enclosing the pair of electrodes. At least one of the electrodes comprises an electrode core coated with a metal coating, the metal coating has a patterned surface structure derived from a patterned surface of the electrode core, and the microfluidic chamber has an inlet for accepting a blood sample comprising platelets for the platelet function test. The measured electrical impedance characterizes platelet aggregation in the blood sample. Such micro platelet function test methods and devices provide a low-cost, micro electrical-mechanical system based testing platform that enable accurate and repeatable platelet function analyses.

### REFERENCE TO RELATED APPLICATIONS

This application claims the benefit of priority to U.S. Ser. No. 62/416,247, filed on 2 Nov. 2016, entitled “An Integrated Miniaturized Platelet Function Analyzer,” the entire disclosure of which is incorporated by reference in its entirety herein.

### FIELD OF THE INVENTION

Embodiments of the present invention relate to the field of platelet function assessment and testing, and pertain particularly to platelet function test devices for platelet impedance aggregometry, and methods for manufacture and use of such devices.

### BACKGROUND OF THE INVENTION

Platelet aggregation plays a key role in normal hemostasis and disturbances in their function can cause life-threatening clotting or bleeding. Platelet function assays (PFAs) are often called upon in a variety of contexts ranging from rendering diagnoses of high impact conditions such as inherited or acquired platelet dysfunctions, monitoring the response to antiplatelet treatments, and ascertaining cardiovascular risks.

Once relegated to tests performed in clinical labs requiring expert interpretation, the promise of using PFAs for the millions of individuals impacted by platelet dysfunctions and cardiovascular diseases drove the development of specialized point-of-care units. Traditional, well-known laboratory tests of platelet function include Bleeding Time (BT), optical aggregometry such as light transmission aggregometry on platelet-rich plasma, lumiaggregometry, impedance aggregometry on whole blood, platelet flow cytometry, and platelet number counting pre- and post-activation in whole blood. Such systems are generally complex, labor intensive, expensive, and require dedicated laboratory setup and standardization.

More recently, simpler point-of-care (POC) testing devices have been made available for platelet function testing, including Multiple Electrode Aggregometry (MEA), Thromboelastography (TEG) platelet mapping, Closure Time evaluation (PFA-100/200), turbidimetric-based optical detection of activated platelets (VerifyNow), and several others. While such POC platelet function testing devices are comparably easier to use, and employ whole blood without sample processing, several major drawbacks remain in their applications to clinical practices. For one, relatively large amounts of blood samples are often required to comprehensively evaluate global and particular aspects of platelet functions. For two, these POC devices often face different issues in terms of device variability and reliability, and test quality control, leading to non-satisfactory clinical outcomes. Recent technologies using impedance aggregometry employ electrodes with non-repeatable surface conditions caused by the manufacturing process of punching or related methods, and cartridge assemblies with device variances caused by the macro-scale manufacturing process. As platelet aggregation is sensitive to electrode surface morphology on the micrometer scale, large and unpredictable variations in testing results often exist. In addition, currently impedance aggregometry lacks automation in general and still requires manual pipetting, which often leads to additional variations in testing results.

Another factor contributing to the limitations of both traditional and recent POC platelet function tests is that in vivo, the relevance of platelets in clot formation is most apparent in arterial flow environments, yet most clinical tests of platelet function fail to consider the central role flow plays in dictating a composite biophysical response. While diagnostic tests often focus on minimizing testing variations caused by physical device and biological patient variation, few clinical-grade tests consider the joint biophysical relationship between platelet and testing environment in a calibrated, controlled, optimized way.

Therefore, in view of the aforementioned difficulties, there is an unsolved need for methods, systems, and devices for platelet function tests that utilize minimal amount of blood while providing highly accurate, reliable, and repeatable test results that allow their use in clinical settings such as in the diagnosis and treatment of bleeding disorders, cardiovascular diseases, trauma coagulopathy, and in transfusion medicine.

It is against this background that various embodiments of the present invention were developed.

### BRIEF SUMMARY OF THE INVENTION

Embodiments of the present invention relate to a low-cost, impedance-based, integrated miniaturized platelet function analysis device, and methods of manufacture and use thereof. Such a micro platelet function test device as described herein use advanced micro-fabrication technologies typically applied in semiconductor device processing and microfluidic principles to attain high levels of control over the device fabrication process while realizing low device cost and achieving high reliability and repeatability in ensuing diagnostic results, as well as the capability to acquire clinical relevant platelet aggregation data under different blood flow conditions.

Accordingly, in one aspect, one embodiment of the present invention is an impedance-based micro platelet function test device, comprising a first pair of micro-fabricated 3D electrodes for measuring an electrical impedance between the first pair electrodes during a platelet function test, and a first microfluidic chamber enclosing the first pair of electrodes. At least one of the electrodes comprises a first electrode core coated with a first metal coating; the first metal coating has a patterned surface structure derived from a patterned surface of the first electrode core; and the first microfluidic chamber has a first inlet for accepting a blood sample comprising platelets for the platelet function test.

In some embodiments, the first microfluidic chamber is a forward microfluidic channel having an outlet for discharging the blood sample, and the blood sample traverses the microfluidics channel uni-directionally as a blood flow from the first inlet to the outlet. In some embodiments, a smallest distance between each of the electrodes and walls of the forward microfluidic channel is at most 1 millimeter. In some embodiments, the forward microfluidic channel comprises a constricted portion and a non-constricted portion, a cross-sectional area of the constricted portion perpendicular to the blood flow is smaller than a cross-sectional area of the non-constricted portion perpendicular to the blood flow, and the first pair of electrodes is positioned within or after the constricted portion in the direction of the blood flow.

In some embodiments, a top-view cross-sectional area of one of the first pair of electrodes, parallel to the blood flow, is non-circular. In some embodiments, a cross-sectional area of one of the first pair of electrodes changes over a height of the electrode. In some embodiments, the surface structure of the first metal coating is controlled between 10 nanometers and 100 micrometers. In some embodiments, the patterned surface structure of the metal coating is non-random. In some embodiments, the at least one of the first pair of electrodes further comprises a second electrode core.

In some embodiments, the micro platelet function test device further comprises a second pair of micro-fabricated 3D electrodes for measuring an electrical impedance between the second pair of electrodes during the platelet function test, where at least one of the second pair of electrodes comprises a second electrode core coated with a second metal coating. In some embodiments, the second pair of electrodes has a configuration different from the first pair of electrodes, and where the configuration is selected from the group consisting of electrode diameter, electrode height, electrode separation, electrode cross-sectional area, and electrode surface structure. In some embodiments, the micro platelet function test device further comprises a second microfluidic chamber enclosing the second pair of electrodes, where the second microfluidic chamber has a second inlet for accepting the blood sample during the platelet function test.

In some embodiments, the micro platelet function test device further comprises a return microfluidics channel, where the return microfluidics channel does not intersect with the forward microfluidic channel, and where the return microfluidics channel connects the outlet and the first inlet to enable a recirculation of the blood flow through the forward microfluidic channel.

In some embodiments, the micro platelet function test device further comprises a first reagent chamber for mixing a first portion of the blood sample with a first platelet-modifying reagent to modify platelet function, where the first reagent chamber comprises a first blood inlet for accepting the first portion of blood sample, a first chamber body where the mixing with the first platelet-modifying reagent occurs, and at least one mixture outlet connected to the inlet of the first microfluidic chamber, and where the first platelet-modifying reagent is pre-loaded in the first chamber body. In some embodiments, the micro platelet function test device further comprises a second reagent chamber for mixing a second portion of the blood sample with a second platelet-modifying reagent to modify platelet function, where the second reagent chamber comprises a second blood inlet for accepting the second portion of the blood sample, a second chamber body where the mixing with the second platelet-modifying reagent occurs, and at least one mixture outlet connected to the inlet of the first microfluidic chamber, where the second platelet-modifying reagent is pre-loaded in the second chamber body, and where the second platelet-modifying reagent is different from the first platelet-modifying reagent.

In some embodiments, the micro platelet function test device further comprises a pump for pumping the blood sample through the first inlet into the first microfluidic chamber, an impedance measurement unit connected to the first pair of electrodes for measuring the electrical impedance between the first pair of electrodes during the platelet function test, and a controller connected to the pump and the impedance measurement unit, for controlling a flow rate of the blood sample in the first microfluidic chamber, and for processing the measured electrical impedance between the first pair of electrodes to characterize platelet responses in the blood flow. In some embodiments, the electrical impedance between the first pair of electrodes is measured continuously as the blood flow circulates through the microfluidic chamber.

In another aspect, one embodiment of the present invention is a method for performing an impedance-based platelet function test using a micro platelet function test device, comprising the steps of injecting a blood sample comprising platelets for the platelet function test into a microfluidic chamber, and measuring an electrical impedance between a first pair electrodes during the platelet function test, where the microfluidic chamber has an inlet for accepting the blood sample, where the microfluidic chamber encloses the first pair of micro-fabricated 3D electrodes, where at least one of the electrodes comprises a first electrode core coated with a first metal coating, and where the first metal coating has a patterned surface structure derived from a patterned surface of the first electrode core.

In some embodiments, the microfluidic chamber is a forward microfluidic channel having an outlet for discharging the blood sample, and the blood sample traverses the microfluidics channel uni-directionally as a blood flow from the first inlet to the outlet. In some embodiments, wherein the surface structure of the first metal coating is controlled between 10 nanometers and 100 micrometers.

In some embodiments, the method further comprises controlling a flow rate of the blood sample in the microfluidic chamber using a pump. In some embodiments, the method further comprises mixing the blood sample with a platelet-modifying reagent inside a reagent chamber to modify platelet function, where the reagent chamber comprises a blood inlet for accepting the blood sample, a chamber body where the mixing with the platelet-modifying reagent occurs, and at least one mixture outlet connected to the inlet of the microfluidic chamber, and where the first platelet-modifying reagent is pre-loaded in the chamber body.

Yet other aspects of the present invention include the methods and processes comprising the steps described herein, and also include the processes and modes of operation of the systems and devices described herein. Other aspects and embodiments of the present invention will become apparent from the detailed description of the invention when read in conjunction with the attached drawings.

### BRIEF DESCRIPTION OF THE DRAWINGS

Embodiments of the present invention described herein are exemplary, and not restrictive. Embodiments will now be described, by way of examples, with reference to the accompanying drawings. For purposes of clarity, not every component is labeled in every drawing. The drawings are not drawn to scale, with emphasis instead being placed on illustrating various aspects of the techniques and devices described herein.

FIG. 1 shows a schematic of a platelet function testing system using a micro platelet function testing device, according to one embodiment of the present invention.

FIG. 2 shows a schematic of a micro platelet function testing device, according to one embodiment of the present invention.

FIG. 3 shows a perspective view of a micro platelet function testing device, according to one embodiment of the present invention.

FIG. 4A shows a perspective view of a micro platelet function testing device having electrodes positioned inside a constricted portion, according to one embodiment of the present invention.

FIG. 4B shows a perspective view of a micro platelet function testing device having electrodes positioned outside a constricted portion, according to one embodiment of the present invention.

FIGS. 5A and 5B show perspective views of a micro platelet function testing device having micro-fabricated 3D electrodes enclosed by a microfluidic channel, respectively, according to one embodiment of the present invention.

FIG. 6 shows schematics illustrating respective steps of a process flow for fabricating 3D electrodes in a microfluidic channel, according to one embodiment of the present invention.

FIGS. 7A, 7B, 7C, 7D, 7E, and 7F show respective images of controlled surface morphologies, according to one embodiment of the present invention.

FIG. 8 shows a perspective side view of structured 3D electrodes each having a core and a patterned metal coating, according to one embodiment of the present invention.

FIG. 9 shows a perspective cross-section view of a micro platelet function test device with structured 3D electrodes each having a patterned core and a metal coating, according to one embodiment of the present invention.

FIG. 10 shows a perspective cross-section view of a micro platelet function test device with structured 3D electrodes having multi-pillared cores, according to one embodiment of the present invention.

FIG. 11 shows schematics illustrating respective steps of another process flow for fabricating 3D electrodes, according to one embodiment of the present invention.

FIG. 12 illustrate respective electrode configurations based on electrode size, separation, and shape, according to various embodiments of the present invention.

FIG. 13 shows a perspective view of a recirculating blood flow micro platelet function testing device, according to one embodiment of the present invention.

FIG. 14 shows an image of a pair of 3D electrodes microfabricated on an oxidized silicon substrate, according to one embodiment of the present invention.

FIG. 15 shows an image of a pair of multi-core 3D electrodes microfabricated on an oxidized silicon substrate, according to one embodiment of the present invention.

FIG. 16 shows graphs of impedance measurements collected using a micro platelet function test device, according to one embodiment of the present invention.

### DETAILED DESCRIPTION OF THE INVENTION

In the following description, for purposes of explanation, numerous specific details are set forth in order to provide a thorough understanding of the invention. It will be apparent, however, to one skilled in the art that the invention can be practiced without these specific details. In other instances, structures, devices, processes, and methods are shown using schematics, use cases, and/or diagrams in order to avoid obscuring the invention. Although the following description contains many specifics for the purposes of illustration, anyone skilled in the art will appreciate that many variations and/or alterations to suggested details are within the scope of the present invention. Similarly, although many of the features of the present invention are described in terms of each other, or in conjunction with each other, one skilled in the art will appreciate that many of these features can be provided independently of other features. Accordingly, this description of the invention is set forth without any loss of generality to, and without imposing limitations upon, the invention.

Broadly, embodiments of the present invention relate to a low-cost, impedance-based, integrated and miniaturized platelet function analysis devices for use in clinical, research, and Point-of-Care (POC) settings, and methods of manufacture and use thereof. Such micro platelet function test devices use advanced micro-fabrication technologies typically applied in semiconductor device processing and microfluidic principles to enable highly accurate, repeatable, and robust platelet function measurements from a very small blood sample.

More specifically, various embodiments of the present invention comprise micro-fabricated 3D electrodes for measuring electrical impedances as activated platelets aggregate on the surface of the electrodes and behave as an electrical insulator. Such micro-fabricated 3D electrodes may be enclosed in a microfluidic chamber, into which a small blood sample activated with a platelet function modifying reagent is injected and circulated. Platelet activation and blood recirculation through the microfluidic chamber may be enabled by the use of interconnected reagent chambers, micro-pumps and micro-valves, while the impedance measurement process may be monitored via a microcontroller.

One key feature of the present invention is in the micro-scale miniaturization and micro-electro-mechanical system design of the platelet function testing devices, which bring important advantages in terms of cost, form factor, disposability, precise control of device geometry and surface morphology, device reproducibility, test automation, and control of the sample shear rate on electrode surface, while also significantly reduce the amount of blood sample required for platelet function testing. Embodiments of the present invention may be mass-produced and implemented as disposable modules ready for integration into cartridge devices or analyzers for different application scenarios, all with well-controlled device variations that provide better diagnostic accuracy and repeatability, enabling multiplexed testing for comprehensive platelet function analysis. For example, standard and specialty hemostasis assays based on the micro platelet function testing device disclosed herein may be used in isolation, in serial, or in a parallelizable fashion at a fraction of the cost and footprint of current stand-alone systems.

Furthermore, embodiments of the present invention leverage the impact of the micro-scale environment on platelet function modification, adhesion, and aggregation, and exploit the role of local fluid shear, by utilizing a novel electrode design where the use of patterned semiconductor, polymer, or metal cores enables precise layout and/or formation of patterned surface structures on metal coatings that cover the electrode cores. In addition, controlled circulation of the blood sample through the microfluidic chamber well mimics the in-vivo biophysical environment of the circulatory system. Compared with existing macro-scale impedance-based devices that suffer from high device variations and largely neglect the relationship between platelet function, shear, and the testing environment, embodiments of the present invention take such factors into account and allow multiscale, multi-stage optimization to create tunable micro platelet function tests that offer new clinical information, enable relational testing of platelet functions in the broader context of hemostasis response without the need for multiple bulky instruments, while maximizing biological and clinical performance.

In the following description, for purposes of explanation, numerous specific details are set forth in order to provide a thorough understanding of the invention. It will be apparent, however, to one skilled in the art that the invention can be practiced without these specific details. In other instances, structures, devices, processes, and methods are shown using schematics, use cases, and/or diagrams in order to avoid obscuring the invention. Although the following description contains many specifics for the purposes of illustration, anyone skilled in the art will appreciate that many variations and/or alterations to suggested details are within the scope of the present invention. Similarly, although many of the features of the present invention are described in terms of each other, or in conjunction with each other, one skilled in the art will appreciate that many of these features can be provided independently of other features. Accordingly, this description of the invention is set forth without any loss of generality to, and without imposing limitations upon, the invention.

### Impedance-Based Micro Platelet Function Testing Device

While optical aggregometry such as light transmission aggregometry on platelet-rich plasma has been viewed as the gold standard in plate function testing over the past few decades, whole blood impedance aggregometry has gained considerable traction in recent years as it allows the assessment of platelet function in anti-coagulated whole blood, without sample processing, under more physiological conditions where the presence of other blood elements may also affect platelet function. Platelet impedance aggregometry is based on the principle that activated platelets adhere to artificial surfaces of electrodes placed within the whole blood sample; aggregated platelets act as an insulator to increase the electrical impedance measurable between a pair of electrodes.

For example, in some impedance aggregometry methods, a very small AC electric voltage in the millivolt range is passed between two electrodes made by precious metal in anti-coagulated whole blood. Such a whole blood sample is placed in a sample chamber heated to 37° C., and may be stirred by magnetic stir bars. Since whole blood is used, the entire platelet population is tested. Upon initial contact with blood, the electrodes become coated with a monolayer of platelets, resulting in a stable impedance value. This stable baseline impedance may be assigned a value of zero ohms of resistance. When an agonist is added, platelets aggregate on the monolayer to increase the impedance, which can be recorded and analyzed. Results of impedance aggregation tests may be qualified by ohms of aggregation at a given time in the test, slope or rate of the reaction in ohms change per minute, or maximum extent of aggregation in ohms. An increase in impedance is directly proportional to the mass of the platelet aggregate. Platelet aggregation in blood is not dependent on the optical characteristics of the sample, so tests may be performed on lipemic and thrombocytopenic samples. In addition, as centrifugation is not required, impedance aggregation is especially useful in conditions where megathrombocyte count is increased. The impedance method allows the study of platelets in the more physiologically representative whole blood environment. Sample preparation is greatly simplified, and preserves labile modulators such as prostacyclin and thromboxane A2, resulting in a testing environment proven to be more sensitive to the effects of many anti-platelet drugs including aspirin, dipyridamole, abciximab, clopidogrel, ticagrelor, ticlopidine, and prasugrel.

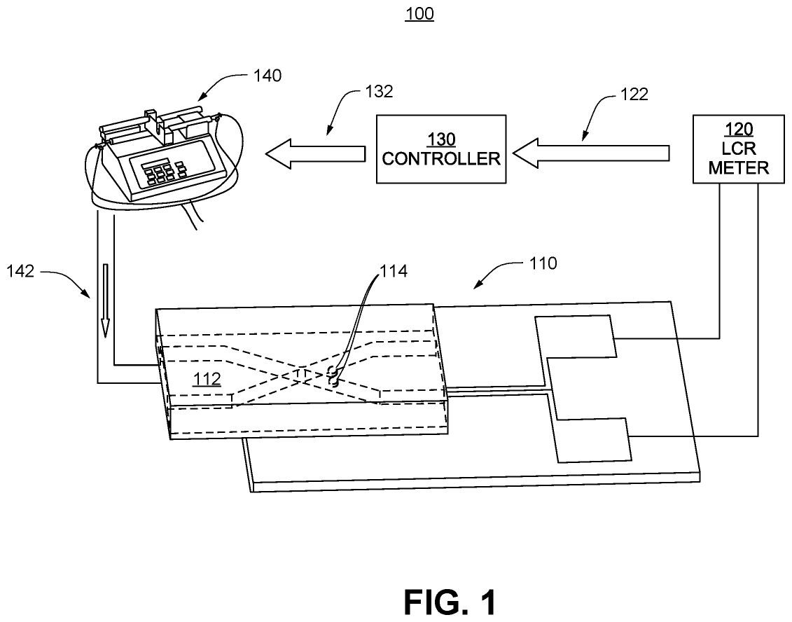

FIG. 1 shows an exemplary schematic of an impedance-based platelet function testing system or analyzer 100 using a micro platelet function testing device 110, according to one embodiment of the present invention. In this example, laboratory-scale components are provided for illustrative purposes only, and further integration into a lab-on-a-chip may be performed in other embodiments of the present invention.

In the impedance-based platelet function testing system 100 shown in FIG. 1, a pump 140 controls the passage of a blood sample 142 into micro platelet function testing device (μ-PFT) 110. While shown as an external syringe pump in FIG. 1, pump 140 may alternatively be a peristaltic pump or a miniaturized integrated micro-pump such as a membrane actuation pump, and may comprise an embedded micro-controller for controlling a volumetric flow rate of the blood sample into μ-PFT device 110. The blood sample may be whole blood, components thereof, platelet-rich plasma, or a mixture of patient sample and one or more reagents such as ADP, and may comprise platelets for function testing.

During platelet function testing, the blood sample flows through a microfluidic chamber 112 enclosing a pair of electrodes 114. Electrodes 114 may be micro-fabricated 3D electrodes integrated into microfluidic chamber 112 with means of exposing them to the controlled blood flow. Platelets aggregate on the electrode surface and change the impedance between the electrodes. Such impedance changes represent the capability of platelets to aggregate, and may be recorded for numerical and diagnostic analyses. Impedance changes across electrodes 114 may be measured using an impedance measurement unit such as a LCR meter 120, or a customized impedance measurement circuit integrated with μ-PFT device 110.

Pump 140 and LCR meter 120 may be jointly controlled by a controller 130, where singular or continuous logging of impedance measurements at discrete time intervals may be carried out while the blood sample is circulated through microfluidic chamber 112 at desired flow rates, shear rates, or other appropriate parameters values. For example, impedance measurements may be made repeatedly over a 10 minute interval, and an area under the ensuing impedance curve may be used to indicate an overall change in impedance value. Measurement data 122 may be transmitted to, recorded, and/or analyzed by controller 130, while control data 132 may be sent to pump 140 for pump parameter configuration. In different embodiments, controller 130 may be an external stand-alone device such as a computer, a hand-held device, or an integrated microcontroller. In some embodiments, controller 130 may further comprise storage memory, network interfaces for communicating with other computers coupled to a network, and user interfaces including inputs and outputs for communicating control and impedance measurement information externally.

In some embodiments of the present invention, microscale surface roughness and topology of micro-fabricated 3D electrodes 114 may be precisely controlled via the use of a semiconductor, polymer, or metal electrode core, covered by a metal coating, formed using advanced semiconductor fabrication techniques. Macroscale geometric electrode parameters such as number, shape, size, and separation may also be configured, to maximize the clinical performance of system 100.

While shown as a unidirectional channel having an inlet and an outlet in FIG. 1 in some embodiments, microfluidic chamber 112 may be a small cell, cavity, or cuvette with a single opening, and an externally-controlled stirring bar may be used to establish a blood flow within the microfluidic chamber. The shear rate of the fluid and its local rate or change near the electrode surface both may be tunable to control shear-dependent platelet aggregation on electrode surfaces.

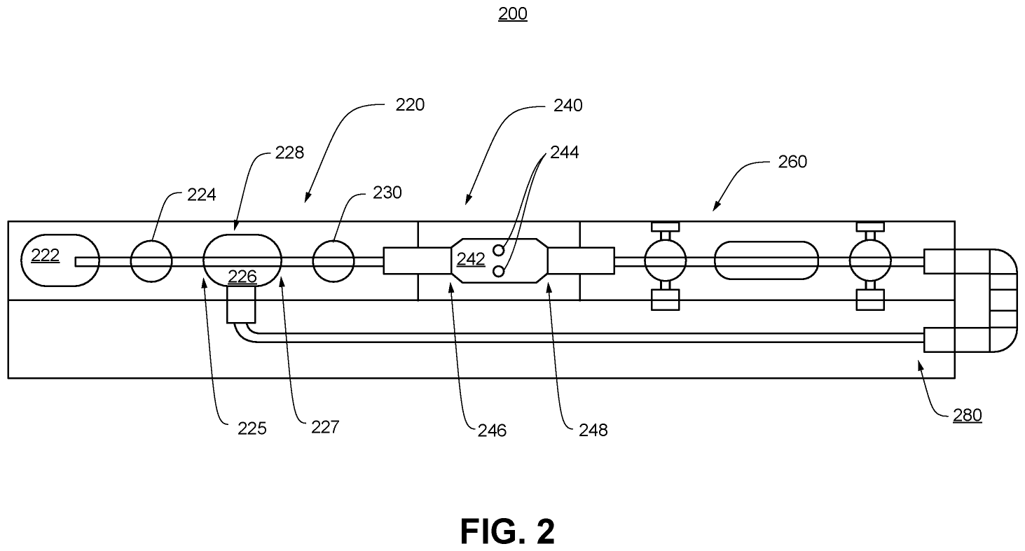

FIG. 2 shows a schematic of an integrated micro platelet function testing device 200, according to one embodiment of the present invention. In this embodiment, μ-PFT device 200 comprises an input module 220, a sensing module 240, a micro-pump module 260, and a recirculation module 280, connected serially in a microfluidic loop to allow a blood sample to be accepted and to flow through electrodes within sensing module 240. In some embodiments, one or more of input module 220, micro-pump module 260, and recirculation module 280 may be optional. In some embodiments, micro-pump module 260 and sensing module 240 may be exchanged in order in this integrated design.

In some embodiments, input module 220 may comprise an optional blood input chamber 222, a reagent chamber 228, and one or more optional micro-valves such as 224 and 230 to regulate blood flow within input module 220. Reagent chamber 228 may be used to mix the blood sample with a platelet-modifying reagent to modify platelet function. In the embodiment shown in FIG. 2, reagent chamber 228 comprises an inlet in the form of an opening 225 for accepting the blood sample, a mixture outlet in the form of opening 227 connected via micro-valve 230 to sensing module 240, and a chamber body 226 in-between, where the mixing of the blood sample with the platelet modifying reagent occurs. In some embodiments, one or more platelet-modifying reagents may be preloaded into reagent chamber 228, either within chamber body 226, or within a separate storage chamber connected to chamber body 226. For example, a platelet-modifying reagent may be preloaded into the chamber body 226 in powder form. Under proper microfluidic pressure configurations, the blood sample may flow into the chamber body and react with the platelet-modifying reagent. In some embodiments, reagent chamber 228 and other components of input module 220 may be made of materials that neither cause platelet adhesion to internal surfaces of input module 220, nor affect platelet aggregation.

In some embodiments, reagent chamber 228 may comprise multiple mixture outlets, each connected to a separate sensing module, possibly under the control of individual micro-valves. Such distribution of the same blood sample to separating sensing modules having different microfluidic chamber and electrode designs allows parallel testing under different microscale environments.

In some embodiments, input module 220 may comprise more than one reagent chambers connected in series or in parallel, with each reagent chamber loaded with individual sets of reagents that may differ in terms of type and/or concentration. In case of parallel reagent chambers, the blood sample may be divided and distributed into each, where a portion of the blood sample mixes with a desired reagent within each reagent chamber. Each reagent chamber may then be connected to a separate sensing unit, or the same sensing unit if desired.

In different embodiments of the present invention, various types of reagents may be used, and optionally preloaded into reagent chamber 228, to enable measurements of specific functional aspects of platelet aggregation. Exemplary platelet modifying reagents include, but are not limited to, thrombin, thrombin receptor activating peptide (TRAP-6; SFLLRN), adenosine diphosphate (ADP), epinephrine, collagen, collagen related peptide (CRP), arachidonic acid, ristocetin, and von Willebrand Factor. Exemplary inhibiting reagents include, but are not limited to, inhibitory prostaglandins such as prostacyclin, P2Y12 inhibitors such as cangrelor, and GPIIBIIIa inhibitors such as abciximab and eptifibatide. Some reagents may be used in the presence of inhibitors of coagulation to isolate effects of platelets, including thrombin inhibitors such as hirudin and bivalirudin, FXa inhibitors, heparins such as unfractionated heparin and low molecular weight heparin, and calcium dictators such as EDTA and citrate.

In some embodiments, sensing module 240 may comprise at least one pair of micro-fabricated 3D electrodes 244 enclosed by a microfluidic chamber 242. Enclosure of the electrodes by the microfluidic chamber ensures that the electrodes come into contact with, and are partially or fully immersed in the blood sample being tested. As described with reference to FIG. 1, electrodes 244 are physically separated and insulated from each other within microfluidic chamber 242, but electrically connected outside microfluidic chamber 242 via a measurement circuit, for determining an impedance between the electrodes during platelet function testing. In various embodiments, microfluidic chamber 242 may have at least one inlet or opening 246 for accepting blood samples for testing. In this embodiment, microfluidic chamber 242 is a microfluidic channel, also having an outlet or opening 248 for discharging the blood sample. The blood sample thus traverses microfluidic chamber 242 uni-directionally as a blood flow from inlet 246 to outlet 248.

In conventional impedance-based platelet function testing devices, a magnetic stirring bar is often used to cause the flow of blood within a much larger version of microfluidic chamber 242, around electrodes 244. Instead of a miniaturization of such mechanical stirring operations, some embodiments of the present invention utilize micro-pump module 260 to regulate a volumetric flow rate of the blood sample through sensing module 240, and to facilitate recirculation of blood through microchannel 280 back to reagent chamber 228 to achieve programmable shear rates using a very small volume of blood. For example, a diaphragm micro-pump with an inlet valve, an outlet valve, and a piezoelectric driver may be used in some embodiments of the present invention. Recirculation microchannel 280 may take on any appropriate shapes and length to optimized flow and shear control. In some embodiments, microfluidic channel 242 may be viewed as a forward microfluidic channel, which recirculation microchannel 280 may be viewed as a return microfluidic channel. Reaction between platelets and platelet-modifying reagents generally become observable via impedance measurements within minutes of the mixing process, and impedance measurements generally increases as more platelet aggregation occur over a period of time on the minute time scale. In some embodiments, micro-pump module 260 circulates, drives, or pushes the blood-sample forwardly then backwardly, back-and-forth across the electrodes, without the use of a recirculation microchannel.

In some embodiments, the integrated fluidic system including the electrodes may be micro-fabricated and assembled with particular dimensions and/or geometries, such that both the shear rate and rate of change of shear rate experienced by the platelets are either preset or programmable. For example, configurable device parameters may include, but are not limited to, a flow rate Q of the blood sample, a size of the microfluidic channel, presence of a constriction or a constricted portion in the microfluidic channel, shape of the microfluidic channel leading to or away from the constriction, such as triangular, semicircular, hyperbolic and the like, placement of electrodes within the microfluidic channel, placement of electrodes relative to the constriction, and the geometry and surface structure of electrodes. In some embodiments, low shears such as 100/s to high shear rates such as to 10,000/s may be programmable with low volumes of blood by using integrated valves and pumping to set up either a back-and-forth flow or a recirculating blood flow. A variety of pumping mechanisms may be used, for example, integrated with the μ-PFT device or a cartridge, using flexible membranes with controlled actuators. Off-cartridge pumping using commercially available syringe pumps or micro-pump may also be used as an alternative.

As integrated sensing module 240 may be batch fabricated with high precision and low variations on the sub-micrometer scale commensurate with the size of individual platelets, the impact of both macro-scale and micro-scale environment on platelet function modification, adhesion, and aggregation may be defined and appropriately controlled via electrode and microfluidic chamber design.

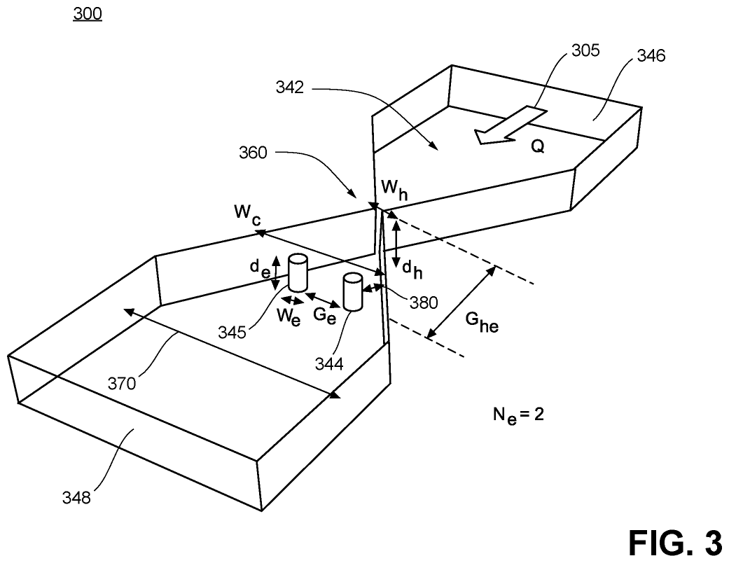

FIG. 3 is a perspective view of a micro platelet function testing device 300, according to one embodiment of the present invention. In this embodiment, μ-PFT device 300 comprises two micro-fabricated 3D electrodes 344 and 345, enclosed by a microfluidic channel 342 having an inlet 346 and an outlet 348, where a blood flow 305 having a volumetric flow rate of Q traverses or passes through microfluidic channel 342 uni-directionally from inlet 346 to outlet 348.

In this embodiment, microfluidic channel 342 has a uniform thickness in the vertical direction, but a constriction or constricted portion 360 in the horizontal direction, where a cross-sectional area of the constricted portion perpendicular to the direction of blood flow 305 is smaller than a cross-sectional area of a non-constricted portion 370, perpendicular to the direction of, or across the blood flow 305. A constricted portion having a narrower or smaller cross-section provides a shear acceleration to achieve a high shear rate in an upstream region immediately before the constriction, while shear deceleration occurs towards the downstream region, where the decreasing shear rate may activate biophysical and biochemical mechanisms to modify platelet function. Electrodes 344 and 345 may be placed in the downstream region, after constricted portion 360 in the direction of blood flow 305.

Several measurement parameters are given in FIG. 3. In particular, the width and height of the constriction or hole 360 in the microfluidic channel are described by wₕ and dₕ. In this embodiment, the cross-section of constriction 360 is rectangular in shape. In other embodiments, the cross-section of constriction 360 may take on square, circular, triangular, or similar shapes. Similarly, a shape of the microfluidic channel leading into and/or out of the constriction may be triangular, semicircular, hyperbolic, or the like, to helps control the shear rate and the shear rate gradient experienced by the blood once introduced into the device. In some embodiments, microfluidic channel 342 may take on non-rectangular cross-sectional shapes along the direction of flood flow 305 as well.

In this illustrative embodiment, electrodes 344 and 345 are cylindrical in shape, positioned on a lower surface of microfluidic channel 342, with a circular top-view cross-section parallel to the direction of blood flow 305. The total number of electrodes is described by Nₑ. A diameter or width of each electrode is described by wₑ, and a height or depth of each electrode is described by dₑ. The width of the gap between the two electrodes are described by Gₑ. A separation between a center of constricted portion 360 and the pair of electrodes is described by Gₕₑ.

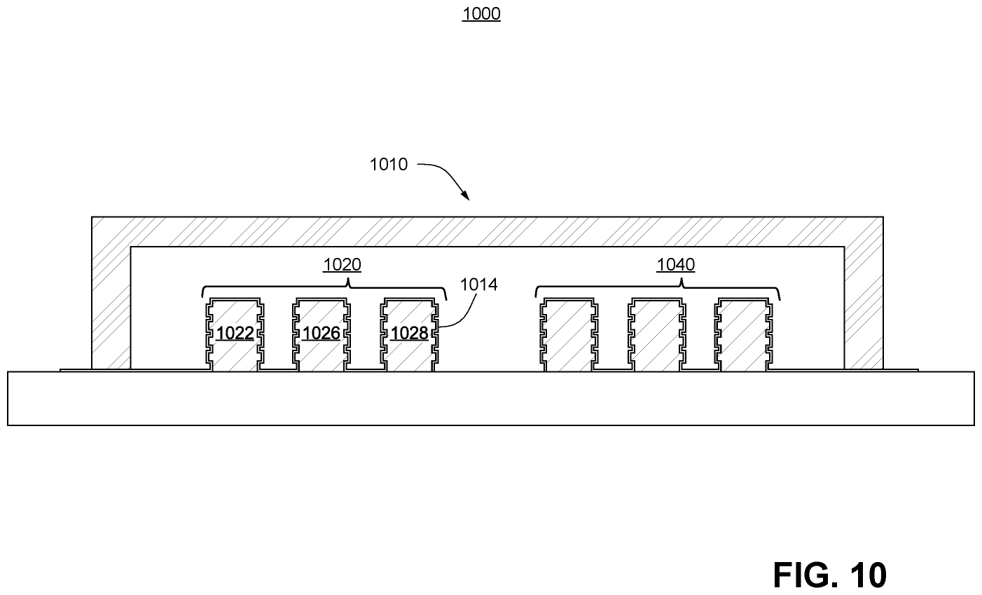

In the present disclosure, an “electrode” refers to a μ-PFT electrode enclosed by a microfluidic chamber, and represents a conductor piece within the microfluidic chamber, electrically separated or insulated from other similar electrodes or conductor pieces. In some embodiments, more than one pair of μ-PFT electrodes may be present, and each pair of electrodes may be configured for separate, parallel impedance measurements across the same blood sample, for example, for average impedance values with higher signal to noise ratios. Thus, Nₑ may be any even integer greater than or equal to 2. In some embodiments, μ-PFT electrodes may be joined or electrically connected outside the microfluidic chamber, for obtaining an impedance measurement across larger electrode surface areas. In such embodiments, Nₑ may be any integer greater than or equal to 2, as any number of μ-PFT electrodes within the microfluidic chamber may be connected externally. Yet in some other embodiments, a single electrode may comprise multiple pillars or multiple cores, where the multiple cores are electrically connected within the microfluidic chamber via a joint metal coating, or similar surface metal connections. An example of multi-core μ-PFT electrodes is discussed with reference to FIG. 10.

In some embodiments, one or both of the electrodes may be non-cylindrical in shape. In some embodiments, one or both of the electrodes may be noncircular cylindrical in shape. For example, a representative cross-section of one or both of electrodes 344 and 345 as viewed from the top may be non-circular. Some exemplary non-circular cross-section shapes include ellipse, rectangle, square, or airfoil.

In some embodiments, cross-sectional areas of the microfluidic chamber and the electrodes in the cross-channel direction, perpendicular to the direction of the blood flow, may be compared, and an absolute value of or a ratio in cross-sectional areas may be bounded to achieve targeted shear rates or rate changes around the electrodes. For example, in FIG. 3, is wₑ represents a width of the channel perpendicular to the direction of the flood flow, along the plane that cut cross the center of the two electrodes, then the ratio between w_{c}dₕ and wₑdₑ may be upper bounded. In some embodiments, a distance between an outer edge of an electrode and the microfluidic channel, represented by (w_{c}−2wₑ−Gₑ)/2, may be upper bounded to less than or equal to 1 mm. In some embodiments, a smallest distance between each of the electrodes and walls of the microfluidic channel, such as distance 380 in FIG. 3, is at most 1 millimeter. In some embodiments, a smallest distance between each of the electrodes and walls of the microfluidic channel is at most 0.5 millimeter.

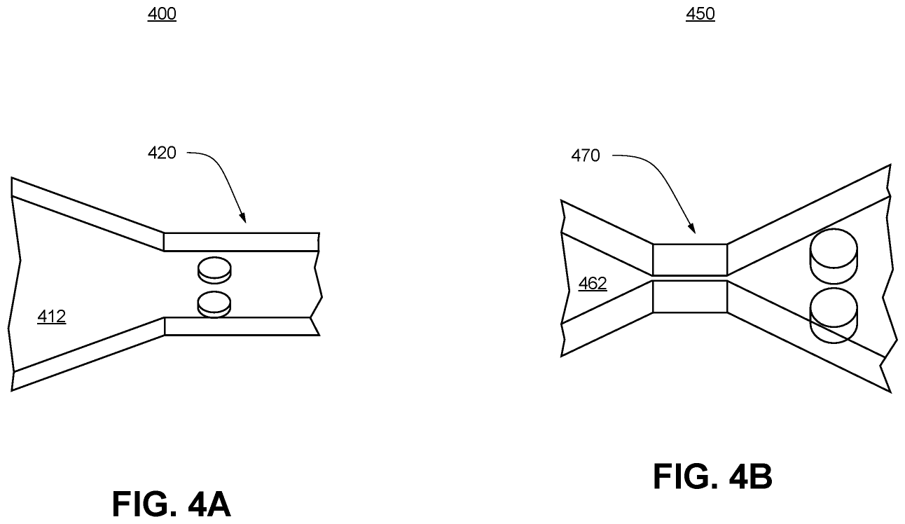

FIG. 4A shows a perspective view 400 of a micro platelet function testing device having electrodes positioned inside a constriction portion, according to one embodiment of the present invention. In this example, microfluidic channel 412 has a constricted portion 420 with a cross-sectional width larger than that of the electrodes. The electrodes are placed within this constricted portion where they experience high blood shear rate in the device. FIG. 4B shows a perspective view 450 of a μ-PFT device having electrodes positioned outside a constricted portion, according to one embodiment of the present invention. In this example, microfluidic channel 462 has a constricted portion 470 with a cross-sectional width smaller than that of the electrodes. The electrodes are placed outside the constricted portion where they experience a high shear rate gradient.

### Electrode Design and Fabrication

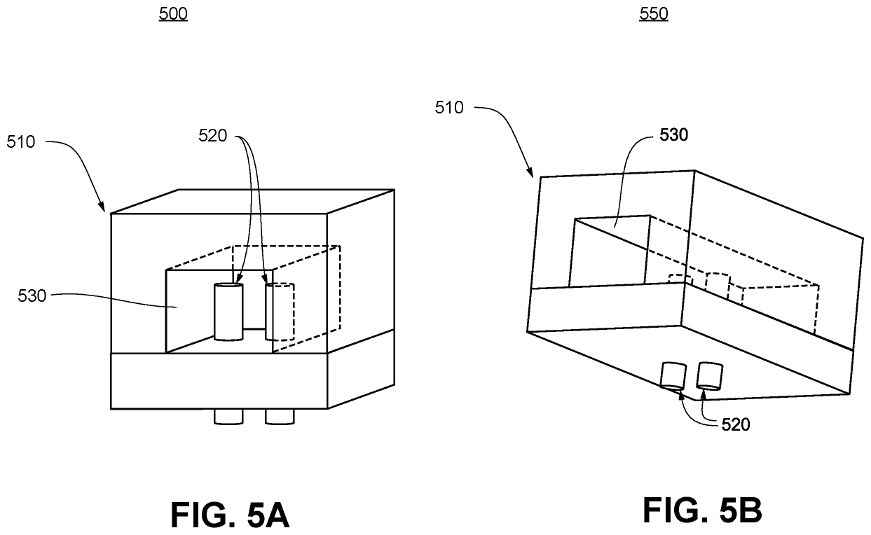

FIGS. 5A and 5B show perspective views 500 and 550 of a micro platelet function testing device 510 having micro-fabricated 3D electrodes 520 enclosed by a microfluidic channel 530, respectively, according to one embodiment of the present invention. In this embodiment, electrodes 520 may be formed by specially treated conductor wires, and embedded or enclosed in microfluidic channel 520 through which the blood sample passes. Such wire electrodes may be made with metals that do not change platelet and coagulation properties, such as Au, Ag, Pt, Cu, and stainless steel. As shown in FIGS. 5A and 5B, the wire electrodes may extend through one side of the microfluidic channel, such as the bottom plastic mechanical support portion, to connect to an external measurement circuit. A length of the electrodes exposed to the channel may be controlled during the fabrication stage. Shorter and thinner electrodes may provide larger signals. In addition, such metal wires may be specially treated to generate a controlled surface roughness, possibly at desired locations, to modify platelet aggregation effect. When view on the micro-scale, such surface roughness may be viewed as random surface structures while a surface structure size distribution statistics is controlled. Both electrochemical and/or mechanical methods may be used to achieved a desired surface roughness. For electrochemical methods, metal wires may be treated with specific chemicals under certain conditions. An example of mechanical method is to use sandpapers to sand metal wire surfaces.

Although not shown explicitly n FIG. 5, embodiments of the present invention may use a novel electrode design where the use of semiconductor, polymer, or pre-patterned metal cores enables precise layout and/or formation of patterned surface structures on finished metal coatings that cover the electrode cores. Exemplary materials for the metal coating include, but are not limited to, Gold (Au), Silver (Ag), Platinum (Pt), Copper (Cu), Titanium (Ti), and stainless steel.

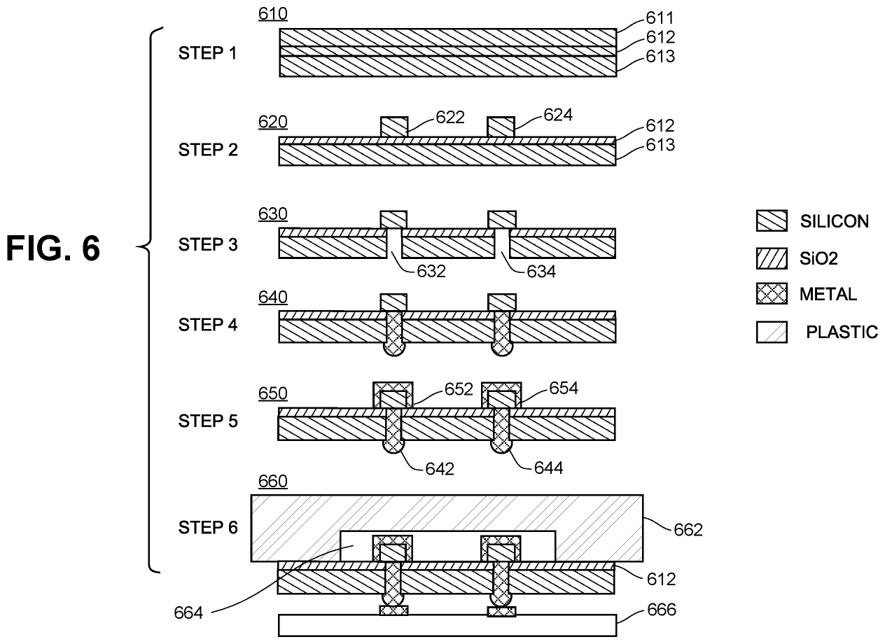

Accordingly, FIG. 6 shows respective schematics illustrating respective steps of a process flow for fabricating such 3D electrodes in a microfluidic channel, according to one embodiment of the present invention. In this embodiment, a silicon-on-insulator (SOI) wafer serves as the starting point for fabrication, and silicon electrode cores are formed using deep reactive-ion etching (DRIE) techniques. Desired patterns may be introduced on the surface of the Si core. Through-silicon via (TSV) technology may then be employed to provide electrical connection to the 3D electrodes from the back of the wafer. Seedless electroplating may then be introduced to produce metal covering the Si core.

More specifically, in a first step 610, the process starts with a double silicon layer wafer with a SiO₂ layer 612 separating two Si layers 611 and 613. Such a double silicon layer wafer may be either a SOI structure or two wafers bonded together. In the latter case, SiO₂ layer 612 may be either thermally grown or CVD grown, which may be followed by annealing to increase quality. The thickness of the two silicon layers may be varied as desired. Bottom layer 613 is a mechanical support layer, which might range, for example, from 300 μm to 800 μm in thickness. Top layer 611 is used for fabricating electrodes and may be the same thickness as the desired electrode length or height, which may range from 1 μm to 5000 μm. If very long electrodes such as greater than 700 μm are needed, bonded silicon wafers may be used as top layer 611. Additionally, top layer 611 may be highly doped to enhance its conductivity. In some embodiments, degenerate doping may be used.

In a second step 620, top layer 611 is etched to form electrode cores 622 and 624 using a photolithography defined mask. This mask may be made of a photoresist, SiO2, SiNx, or even metal layer such as Gold (Au), Aluminum (Al), and Silver (Ag). Exemplary etching processes include dry etching techniques such as deep reactive ion etching.

In a third step 630, a TSV process may be performed while keeping the two electrode cores electrically separated. When electrode cores 622 and 624 are not very small, such as above 50 μm in height, many existing TSV processes may handle such sizes, if a desired electrode size is very small, such as approximately 20 μm, mechanical support layer 611 may be thinned down, for example, to the scale between 100 μm to 200 μm first.

In a fourth step 640, two holes 632 and 634 are etched to open the backside of the electrode, including the SiO₂ underneath the electrode. In step 640, metal is filled into the holes to form connections 642 and 644 to the electrodes.

In a fifth step 650, surfaces of electrode cores 622 and 624 are coated with a metal suitable for platelet aggregometry, including but not limited to, Au, Ag, Cu, and Pt. In some embodiments, electroplating techniques such as seedless electrical plating may be used in this step to form metal coatings 652 and 654. In some embodiments where a seed layer is used, a shadow mask may be used to ensure that only the area around the two electrode cores is coated with seed metal layers. In some embodiments, alternative techniques such as e-beam deposition, sputtering, and other CVD methods may be used.

In a sixth step 660, a plastic or Polydimethylsiloxane (PDMS) layer 662 with a build-in blood flow channel 664 is bonded to SiO₂ surface 612. This bonding may be achieved through plasma bonding or the application of an adhesive. On the backside of the silicon wafer, additional electrical connections may be formed, for example by the use of a printed circuit board 666, to connect the two electrodes to impedance measurement circuits.

In this embodiment, the microfabrication process as described above not only allows the formation of 3D electrodes having desired shape, size, and configurations, it also enables the creation of flat surfaces around the electrode for bonding with plastic materials for the formation of the microfluidic channel. In additional, while microfluidic channel 664 is “sealed” to prevent fluid leakage, the metal-coated electrodes enclosed in the microfluidic channel are still electrically accessible externally.

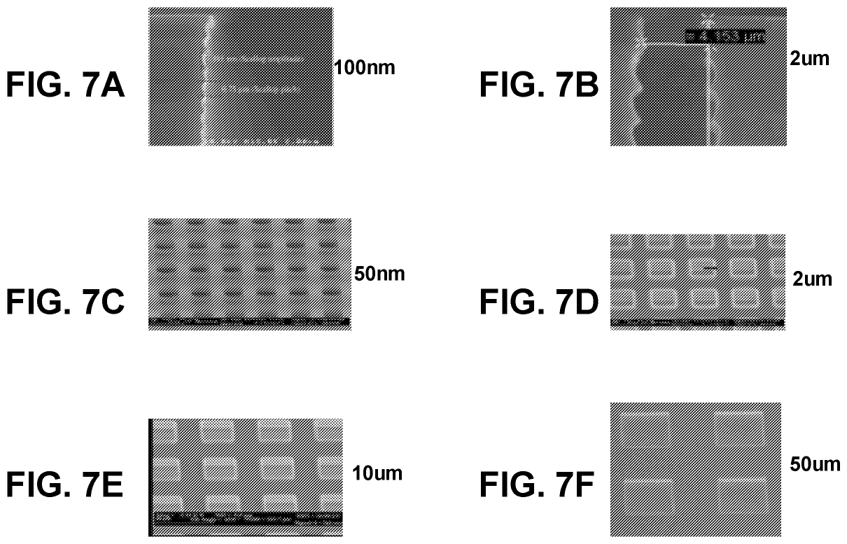

FIGS. 7A and 7B, 7C, 7D, 7E, and 7F show respective images of controlled surface morphologies, according to one embodiment of the present invention. Electrode surface structures may significantly modify platelet adhesion behavior. For example, when metal wires formed by pulling are roughed with sand papers of different grades, systematic changes in surface roughness may result in significantly different platelet coating patterns. In the present disclosure, surface roughness refers to the unevenness and irregularities of a material surface, and a patterned surface refers the case where surface roughness is controlled or limited in size, convexity, and/or location. A patterned surface may comprise random surface structures with a certain desired statistical distribution of surface structure parameters including limitations on dimensions. A patterned surface may also comprise non-random or designed regular patterns with configurable parameters. By micro-scale surface patterning and topology engineering, more platelets may aggregate on the electrode body, and undesired coverage heterogeneity caused by flow and geometry may be compensated. Such surface patterning processes are facilitated by the novel electrode design using easily patterned semiconductors or polymer cores, or pre-patterned metal cores.

FIG. 7A shows an image of an exemplary etched electrode core sidewall having deep reactive ion etching (DRIE) induced scallops with amplitudes and pitches within the 100 nm range. FIG. 7B shows an image of another exemplary electrode core sidewall having scallop amplitudes and pitches within the micrometer range. These examples illustrate that DRIE-Induced scallops may be used to optimize surface topology precisely and economically, providing a unique advantage in terms of surface patterning and topology engineering in micro-fabricated electrodes. In different embodiments, scallop periodicity and depth may be controlled independently, and scallop periodicity may vary, depending on the limitations of the fabrication process, for example, between 0.1 μm and 3 μm. While periodic DRIE-induced scallops may be viewed as non-random surface patterns, in some embodiments, non-periodic surface patterns may be produced to affect platelet aggregation. For example, in some embodiments, traditional surface roughening techniques such as grinding and sanding may be used to control metal electrode core surface roughness in a random but controlled fashion. Similarly, in some embodiments, electrochemical roughening may be used to control electrode core surface roughness, which then translates to desired surface patterns on the electrode metal coating. In some embodiments of the present invention, the surface structure of the electrode core and/or the metal coating may be controlled between a desired range, such as between 10 nm and 100 μm.

While FIGS. 7A and 7B shows DRIE-induced surface patterns, FIGS. 7C, 7D, 7E, and 7F illustrate uniformly distributed surface patterns that may be realized by DRIE processes, where square protrusions on the scale of 50 nm, 2 μm, 10 μm, and 50 μm are shown respectively. Other repeating or non-repeating patterns may be used in various embodiments of the present invention. For example, horizontal or vertical grooves, spirals, zig-zags, and the like. Again, different electrode surface microstructure or roughness may provide different platelet coating densities, leading to beneficial heterogeneities in the resulting impedance measurements. For example, the diagnostic capability of μ-PFT devices as disclosed herein in separating patient groups having different platelet function responses may be optimized when surface roughness is present, but also bounded within an optimal range.

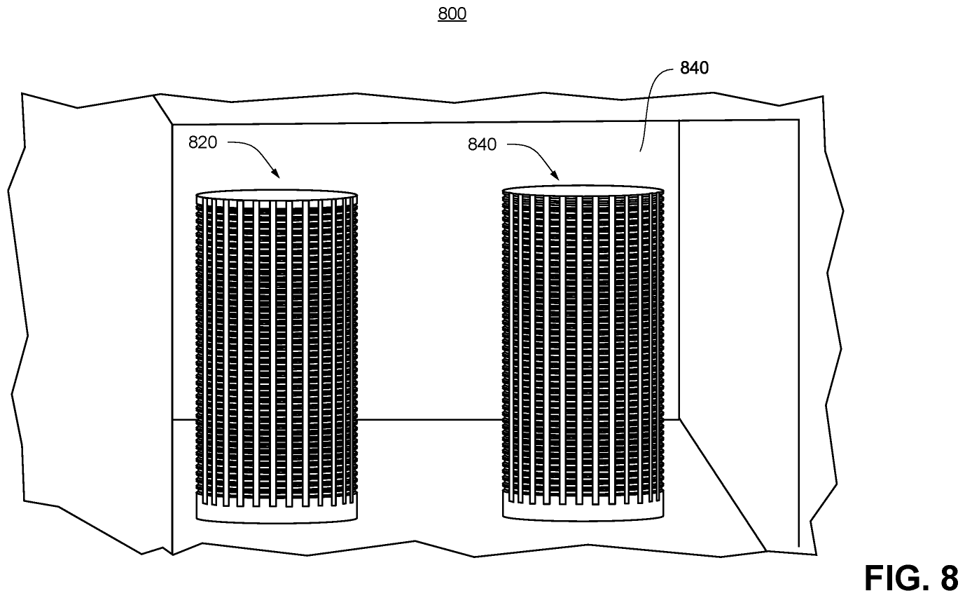

FIG. 8 shows a perspective side view 800 of two structured 3D electrodes 820 and 840 each having a core and a patterned metal coating, according to one embodiment of the present invention. In this embodiment, electrodes 820 and 840 are enclosed in a microfluidic channel 860. Each electrode may have a silicon, polymer, or metal core coated with a metal coating, where the metal coating may have patterned surface structures derived from a patterned surface of the electrode core. For example, each electrode core may have, on the side surfaces, undulating DIRE-induced scallops shown in FIG. 7A or 7B. Furthermore, in this example, electrodes 820 and 840 have identical radially symmetric surface patterns. In some embodiments, surface patterns may be asymmetric on each electrode, and/or the two electrodes may comprise different surface patterns.

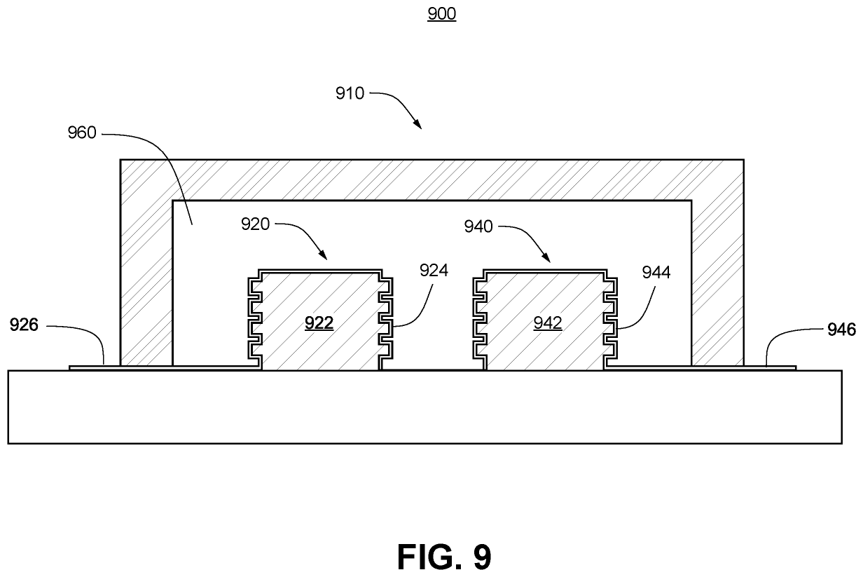

FIG. 9 shows a perspective cross-section view 900 of a micro platelet function test device 910 with structured 3D electrodes each having a patterned core and a metal coating, according to one embodiment of the present invention. In this embodiment, electrodes 920 and 940 are enclosed or encapsulated in a polymer microfluidic channel or microfluidic chamber 960. Electrode 920 has a patterned silicon or polymer core 922 coated with a metal coating 924; electrode 940 has a patterned silicon or polymer core 942 coated with a metal coating 944. The metal coatings have patterned surface structures derived from the patterned surfaces of the electrode cores. The patterned surface structures shown in FIG. 9 are similar to those shown in FIGS. 7C, 7D, 7E, and 7F. Moreover, in this embodiment, metal coatings 924 and 944 extend laterally to form metal electrical connections 926 and 946, respectively.

Similarly, FIG. 10 shows a perspective cross-section view 1000 of a micro platelet function test device 1010 with structured 3D electrodes having multiple cores, according to one embodiment of the present invention. Multi-core electrodes provide more edge adhesion effects for platelet aggregation. In this embodiment, electrode 1020 comprises three patterned electrode core pillars 1022, 1026, and 1028 made of silicon or polymer, and the individual electrode core pillars are jointly coated with a metal coating 1024 to form a single electrode. Electrode 1040 is formed in a similar fashion. While electrode core pillars 1022, 1026 and 1028 are shown to have an equidistance side view, they may be positioned within microfluidic chamber 1060 in any desired positions.

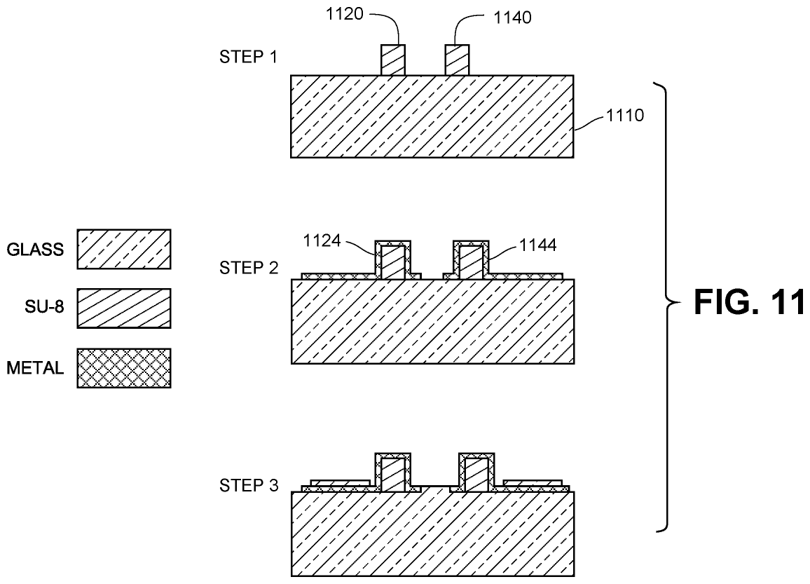

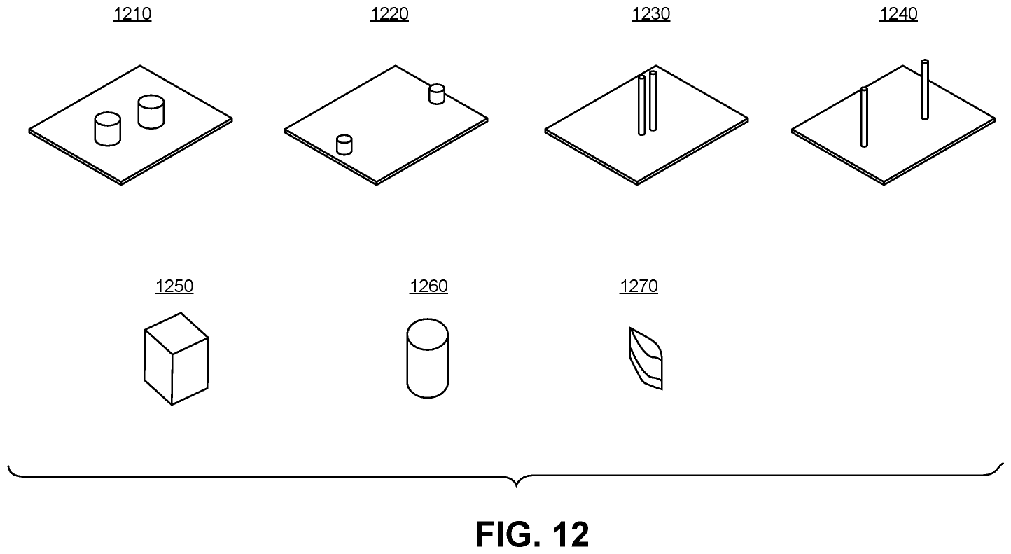

FIG. 11 shows respective schematics illustrating respective steps of another process flow for fabricating 3D electrodes, according to one embodiment of the present invention. Starting with an insulating glass insulating wafer 1110, a photo-sensitive polymer material SU-8 is formed as electrode cores 1120 and 1140 by photolithography. 2D patterns may be introduced on the core surface. A tilted evaporation process may then be performed to cover the SU-8 cores with metal coatings 1124 and 1144 in a second step. In a third step, oxide evaporation may be performed to partially cover the metal layer with a very thin layer of oxide material, to reduce parasitics and enhance the measured signal to noise ratio.

While discussions in electrode design so far has focused on micro-scale considerations in terms of surface roughness and surface structure pattern design, macro-scale factors such as electrode diameter, electrode height, electrode separation, electrode cross-sectional areas, and relative positions of the electrodes with the microfluidic chamber also play important roles in platelet aggregation. Such electrode parameters affect the measurement signal in a non-trivial manner because of their conjugated effect on electrical fields, flow dynamics and biological processes.

FIG. 12 illustrate respective electrode configurations based on electrode size, separation, and shape, according to various embodiments of the present invention. More specifically, the top row in FIG. 12 shows four different cylindrical electrode configurations 1210, 1220, 1230 and 1240, where electrode diameter, length/depth, and separation each differs. The drawings are not drawn to scale, and are for illustrative purposes only. The bottom row in FIG. 12 shows electrodes 1250, 1260, and 1270 having rectangular, circular, and airfoil shaped cross-sections. Average shears at different sections of the electrodes with different shapes differ under the same flow. For example, when shear homogeneity is desirable in some embodiments of the present invention, airfoil shaped electrodes may be used.

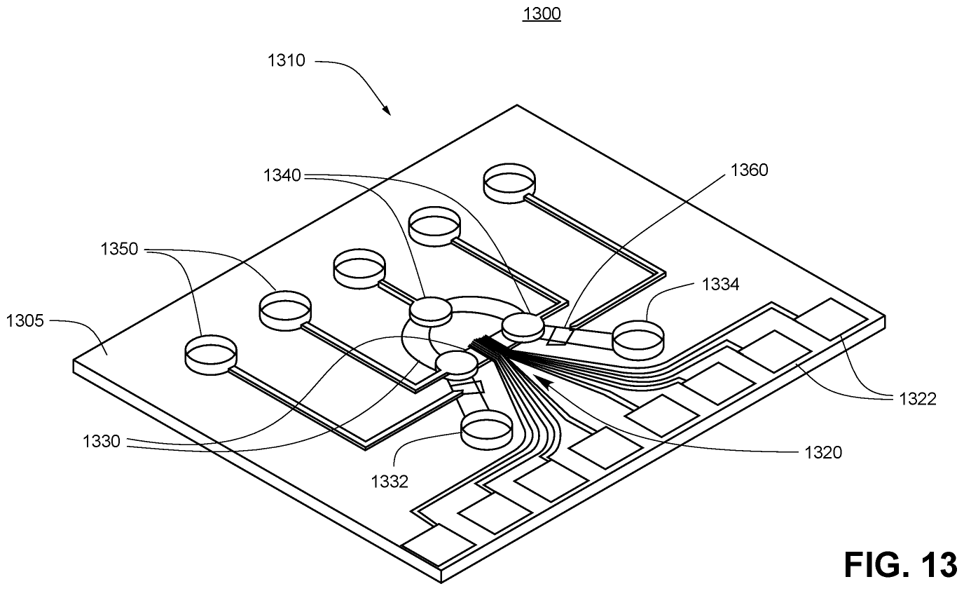

FIG. 13 shows a perspective view 1300 of a recirculating blood flow micro platelet function testing device 1310, according to one embodiment of the present invention. In this embodiment, eight pairs of electrodes 1320 are integrated into a microfluidic channel and chamber system which includes microfluidic channels such as 1330, micro-pneumatic membrane pumps such as 1340, micro-pneumatic membrane valves such as 1360, and pneumatic connections such as 1350. The micro-pneumatic membrane pumps may be actuated using pneumatic controls, allowing for blood flow rate and shear rate control from low to high, for example between 100/s to 5000/s, using a small volume of blood, such as between 1 μL and 10 μL.

### Illustrative Embodiment

Without loss of generality, an illustrative embodiment is now described.

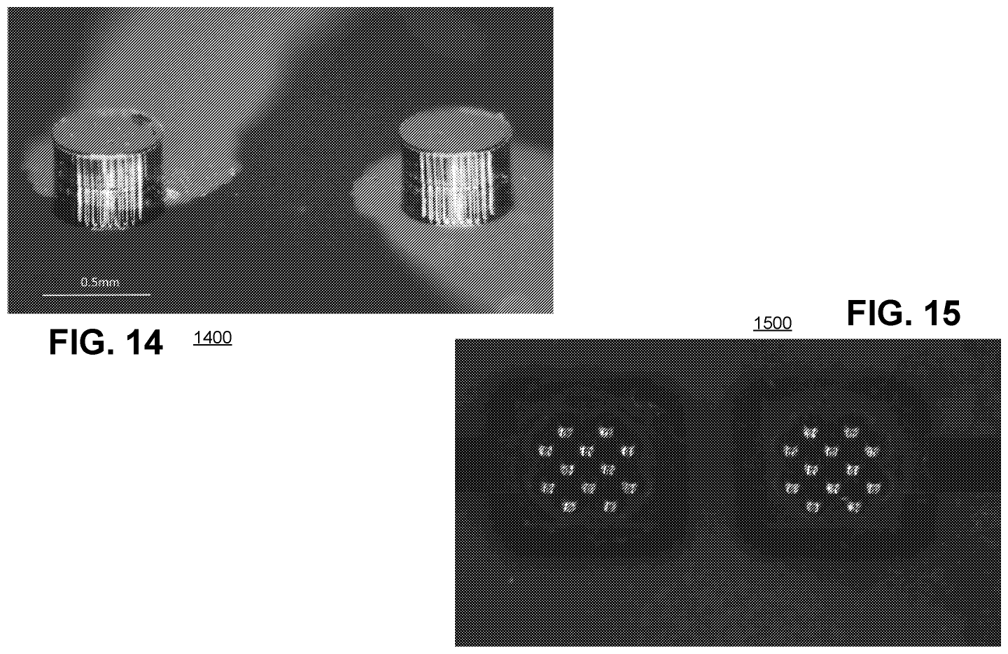

FIG. 14 shows an illustrative image 1400 of a pair of 3D electrodes microfabricated on an oxidized silicon substrate with pre-patterned metal connections and pads, according to one embodiment of the present invention. The 3D electrodes were made with a polymeric core of photo-patterned cross-linked SU-8 and a metal coating comprising a film of gold and an adhesion layer of titanium. The electrodes are cylindrical in shape and have a surface pattern consisting of vertical grooves.

Although not shown explicitly, electrodes of different heights (125 μm, 250 μm), diameters (100 μm, 250 μm, 500 μm), and gaps in-between electrodes (500 μm, 1000 μm) were fabricated using the same process. Pairs of 3D electrodes where each electrode comprises a number of electrically connected metal-coated cylindrical cores arranged in a grid were also fabricated. Each fabricated 3D electrode pair was aligned and assembled with a microfluidic channel made of polydimethylsiloxane (PDMS), which had an internal height of 1 mm and an internal width of 5 mm, and tubing was connected to the inlet and outlet of the PDMS. Wires soldered to the patterned metal pads on the substrates were used to connect the 3D electrodes to an Agilent E4980A LCR meter.

FIG. 15 shows an image 150 of a pair of 3D electrodes where each electrode is made of 12 electrically connected metal-coated cylindrical cores arranged in a grid.

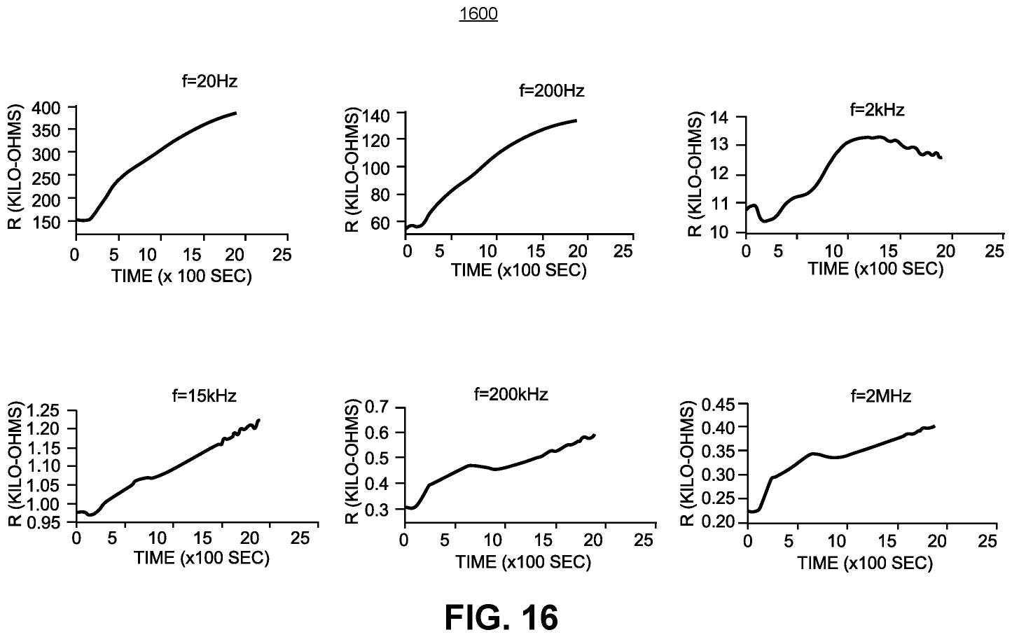

FIG. 16 show a set 1600 of experimental data using electrodes fabricated according to the process discussed above. 1 mL of fresh anticoagulated human blood, obtained within 4 hrs of use, was mixed with 65 μL of 100 mg/mL Calcium Chloride and 10 μM of D-Phenylalanyl-prolyl-arginyl chloromethyl ketone (PPACK). This mixture was flowed into the assembled microfluidic channel with 3D electrodes at a flow rate of 25 μL/min using a syringe pump. The impedance of the electrodes was continuously monitored and logged at six different frequencies (20 Hz, 200 Hz, 1 kHz, 15 kHz, 200 kHz, 2 MHz) using a custom MATLAB program running on a computer interfaced to the LCR meter. Impedance values at multiple frequencies may be used to improve the signal-to-noise ratio of the platelet function measurement. After the blood was flowing through the device, 100 μM of adenosine diphosphate (ADP), a platelet activation reagent, was added. Following this step, increase in electrode impedances at all frequencies may be observed which was monitored and logged for a total time of 2000 s. The peak impedance values reached or the areas under the impedance-time curves or other measures from these curves may be analyzed and interpreted as indications of platelet activity in response to the modifying reagent added (e.g. ADP). Decrease in such measures from normal values may be indicative of non-responsiveness to the reagent and may be used to assess drug-resistance such as clopidogrel resistance. In various embodiments, additional diagnoses may be conducted using a selection of appropriate set of reagents.

One of ordinary skill in the art knows that the use cases, structures, schematics, and flow diagrams may be performed in other orders or combinations, but the inventive concept of the present invention remains without departing from the broader scope of the invention. Every embodiment may be unique, and methods/steps may be either shortened or lengthened, overlapped with other activities, postponed, delayed, and continued after a time gap, such that every user is accommodated to practice the methods of the present invention.

Although the present invention has been described with reference to specific exemplary embodiments, it will be evident that the various modification and changes can be made to these embodiments without departing from the broader scope of the invention. Accordingly, the specification and drawings are to be regarded in an illustrative sense rather than in a restrictive sense. It will also be apparent to the skilled artisan that the embodiments described above are specific examples of a single broader invention which may have greater scope than any of the singular descriptions taught. There may be many alterations made in the descriptions without departing from the scope of the present invention as defined by the appended claims.

What is claimed is:

1. An impedance-based micro platelet function test device, comprising:
a first pair of micro-fabricated 3D electrodes for measuring an electrical impedance between the first pair electrodes during a platelet function test,
wherein at least one of the electrodes comprises a first electrode core coated with a first metal coating, and
wherein the first metal coating has a patterned surface structure derived from a patterned surface of the first electrode core; and
a first microfluidic chamber enclosing the first pair of electrodes, wherein the first microfluidic chamber has a first inlet for accepting a blood sample comprising platelets for the platelet function test.

2. The micro platelet function test device of claim 1,
wherein the first microfluidic chamber is a forward microfluidic channel having an outlet for discharging the blood sample, and
wherein the blood sample traverses the microfluidics channel uni-directionally as a blood flow from the first inlet to the outlet.

3. The micro platelet function test device of claim 2,
wherein a smallest distance between each of the electrodes and walls of the forward microfluidic channel is at most 1 millimeter.

4. The micro platelet function test device of claim 2,
wherein the forward microfluidic channel comprises a constricted portion and a non-constricted portion,
wherein a cross-sectional area of the constricted portion perpendicular to the blood flow is smaller than a cross-sectional area of the non-constricted portion perpendicular to the blood flow, and
wherein the first pair of electrodes is positioned within or after the constricted portion in the direction of the blood flow.

5. The micro platelet function test device of claim 2, wherein a top-view cross-sectional area of one of the first pair of electrodes, parallel to the blood flow, is non-circular.

6. The micro platelet function test device of claim 1, wherein a cross-sectional area of one of the first pair of electrodes changes over a height of the electrode.

7. The micro platelet function test device of claim 1, wherein the surface structure of the first metal coating is controlled between 10 nanometers and 100 micrometers.

8. The micro platelet function test device of claim 1, wherein the patterned surface structure of the metal coating is non-random.

9. The micro platelet function test device of claim 1, wherein the at least one of the first pair of electrodes further comprises a second electrode core.

10. The micro platelet function test device of claim 1, further comprising:
a second pair of micro-fabricated 3D electrodes for measuring an electrical impedance between the second pair of electrodes during the platelet function test, wherein at least one of the second pair of electrodes comprises a second electrode core coated with a second metal coating.

11. The micro platelet function test device of claim 10,
wherein the second pair of electrodes has a configuration different from the first pair of electrodes, and wherein the configuration is selected from the group consisting of electrode diameter, electrode height, electrode separation, electrode cross-sectional area, and electrode surface structure.

12. The micro platelet function test device of claim 10, further comprising:
a second microfluidic chamber enclosing the second pair of electrodes, wherein the second microfluidic chamber has a second inlet for accepting the blood sample during the platelet function test.

13. The micro platelet function test device of claim 2, further comprising:
a return microfluidics channel,
wherein the return microfluidics channel does not intersect with the forward microfluidic channel, and
wherein the return microfluidics channel connects the outlet and the first inlet to enable a recirculation of the blood flow through the forward microfluidic channel.

14. The micro platelet function test device of claim 1, further comprising:
a first reagent chamber for mixing a first portion of the blood sample with a first platelet-modifying reagent to modify platelet function,
wherein the first reagent chamber comprises a first blood inlet for accepting the first portion of blood sample, a first chamber body where the mixing with the first platelet-modifying reagent occurs, and at least one mixture outlet connected to the inlet of the first microfluidic chamber, and
wherein the first platelet-modifying reagent is pre-loaded in the first chamber body.

15. The micro platelet function test device of claim 14, further comprising:
a second reagent chamber for mixing a second portion of the blood sample with a second platelet-modifying reagent to modify platelet function,
wherein the second reagent chamber comprises a second blood inlet for accepting the second portion of the blood sample, a second chamber body where the mixing with the second platelet-modifying reagent occurs, and at least one mixture outlet connected to the inlet of the first microfluidic chamber,
wherein the second platelet-modifying reagent is pre-loaded in the second chamber body, and
wherein the second platelet-modifying reagent is different from the first platelet-modifying reagent.

16. The micro platelet function test device of claim 1, further comprising:
a pump for pumping the blood sample through the first inlet into the first microfluidic chamber;
an impedance measurement unit connected to the first pair of electrodes for measuring the electrical impedance between the first pair of electrodes during the platelet function test; and
a controller connected to the pump and the impedance measurement unit, for controlling a flow rate of the blood sample in the first microfluidic chamber, and for processing the measured electrical impedance between the first pair of electrodes to characterize platelet responses in the blood flow.

17. The micro platelet function test device of claim 1, wherein the electrical impedance between the first pair of electrodes is measured continuously as the blood flow circulates through the microfluidic chamber.

18. A method for performing an impedance-based platelet function test using a micro platelet function test device, comprising:
injecting a blood sample comprising platelets for the platelet function test into a microfluidic chamber,
wherein the microfluidic chamber has an inlet for accepting the blood sample,
wherein the microfluidic chamber encloses a first pair of micro-fabricated 3D electrodes,
wherein at least one of the electrodes comprises a first electrode core coated with a first metal coating, and
wherein the first metal coating has a patterned surface structure derived from a patterned surface of the first electrode core; and
measuring an electrical impedance between the first pair electrodes during the platelet function test.

19. The method of claim 18,
wherein the microfluidic chamber is a forward microfluidic channel having an outlet for discharging the blood sample, and
wherein the blood sample traverses the microfluidics channel uni-directionally as a blood flow from the first inlet to the outlet.

20. The method of claim 18, wherein the surface structure of the first metal coating is controlled between 10 nanometers and 100 micrometers.

21. The method of claim 18, further comprising:
controlling a flow rate of the blood sample in the microfluidic chamber using a pump.

22. The method of claim 18, further comprising:
mixing the blood sample with a platelet-modifying reagent inside a reagent chamber to modify platelet function,
wherein the reagent chamber comprises a blood inlet for accepting the blood sample, a chamber body where the mixing with the platelet-modifying reagent occurs, and at least one mixture outlet connected to the inlet of the microfluidic chamber, and
wherein the first platelet-modifying reagent is pre-loaded in the chamber body.
